(19) United States
(12) Patent Application Publication
(10) Pub. No.: US 2019/0343416 A1
(43) Pub. Date: Nov. 14, 2019

(54) Deriving mean brain frequency as a measure of homeostasis and providing guided interventions to correct thoughts,perceptions,and stimuli effecting the user's neuro signals

(71) Applicants: Graham Boulton, Windaroo (AU); Henry Boulton, Windaroo (AU)

(72) Inventors: Graham Boulton, Windaroo (AU); Henry Boulton, Windaroo (AU)

(21) Appl. No.: 16/406,041

(22) Filed: May 8, 2019

Related U.S. Application Data
(60) Provisional application No. 62/669,394, filed on May 10, 2018.

(51) Int. Cl. A61B 5/0482; A61B 5/00; A61B 5/04

(52) U.S. Cl. CPC A61B 5/0482; A61B 5/4064; A61B 5/04001; A61B 5/7203; A61B 5/04004; A61B 5/6814; A61B 5/0006; A61B 5/04017; A61B 5/6803

(57) ABSTRACT

Disclosed is a method, apparatus and neuro-feedback system for processing neuro-signals of a user's brain to derive mean brain frequency that correspond to a homeostasis condition of the user's Autonomic Nervous System (ANS). The derived mean brain frequency from the neuro-signals or bio signals of the user's brain correspond to Brain Frequency Variability (BFV) index that also correspond to a homeostasis condition of the user's ANS like the mean brain frequency. The disclosed system can be used to train the user's brain using one or more guided interventions in response to the derived mean brain frequency or the BFV index to correct thoughts, perceptions, and stimuli that influences state of the user's neuro-signals.

### CROSS-REFERENCE TO RELATED PATENT DOCUMENTS

This patent application claims the benefit of priority of U.S. Provisional Application No. 62/669,394, entitled “METHOD, APPARATUS AND NEURO-FEEDBACK SYSTEM FOR PROCESSING NEURO-SIGNALS OF A SUBJECTS'S BRAIN TO DERIVE THE BRAIN FREQUENCY HOMEOSTASIS AS A MEASURE OF ANS” filed on May 10, 2018, which is hereby incorporated herein by reference in its entirety.

### FIELD OF INVENTION

The present invention relates generally to a method and apparatus for processing neuro-signals. More particularly, the present invention pertains to a method, apparatus and a neuro-feedback system for processing neuro-signals of a user's brain to derive a mean brain frequency corresponding to a homeostasis condition of the user's Autonomic Nervous System (ANS), and providing guided interventions to the user to correct thoughts, perceptions, and stimuli that influences state of the neuro signals of the user or the ANS.

### BACKGROUND OF INVENTION

The Autonomic Nervous System (ANS) is the portion of the nervous system that controls the body's visceral functions, including action of the heart, movement of the gastrointestinal tract, and secretion by different glands, and many other vital activities, in order to maintain homeostasis of the body. The two major subdivisions of the ANS (i.e., the sympathetic and parasympathetic) regulate the body in response to an ever-changing internal and external environment. The sympathetic system is known as “Fight or Flight” system. It activates the body and mind for exercise and work and it prepares the body to meet real or imagined threats to its survival. The parasympathetic system is known as “Rest & Digest”. When the parasympathetic system is activated, we generally tend to relax, calm and slow down. Both of these sub-systems can have inhibitory effects in some organs and excitatory effects in others. For example, the generally exciting sympathetic system inhibits the digestive musculature and by exciting the micro-vascular arteriolar sphincters it reduces the digestive blood flow. Conversely, the enervating parasympathetic system extraordinarily excites the digestive system to increase the visceral blood circulation.

Homeostasis is exclusively a biological term, referring to the concepts described by Claude Bernard (in 1865) and Walter Cannon (in 1926), concerning the constancy of the internal environment in which the cells of the body live and survive. Homeostasis in users is defined as the stable state of the user and of his internal environment as the maintenance or regulation of the stable condition, or its equilibrium or simply as the balance of bodily functions. The stable condition is the condition of optimal functioning for the user, and is dependent on many variables, such as body temperature and fluid balance, being kept within certain pre-set limits. Other variables include the pH of extracellular fluid, the concentrations of sodium, potassium and calcium ions, as well as that of the blood sugar level, and these need to be regulated despite changes in the environment, diet, or level of activity. Each of these variables is controlled by one or more regulators or homeostatic mechanisms, which together maintain life.

The homeostasis is brought about by a natural resistance to change in the optimal conditions, and equilibrium is maintained by many regulatory mechanisms. All homeostatic control mechanisms have at least three interdependent components, namely receptor, control center, and an effector. The receptor is the sensing component that monitors and responds to changes in the environment, either external or internal. Receptors include thermo-receptors, and mechanoreceptors. The control centres include the respiratory center, and the renin-angiotensin system. The effector is the target acted on, to bring about the change back to the normal state.

The homeostasis and/or the ANS and its role in maintenance of health have been explored in the past. There are several prior art methods and devices that use the concept of applying sensory stimuli to a user's body to affect the user's health or condition. For example: U.S. Pat. No. 4,289,121 discloses a method and device for controlling the functional state of the central nervous system using audio and light signals applied according to the body's biorhythms that correspond to a stable state of the central nervous system. U.S. Pat. No. 4,289,121 describes modulating frequencies depending on the bio-signals or EEG, or the measured respiration rate of the patient, wherein the amplitude or rhythmic signals correspond to the volume of sound and brightness of light. The device as disclosed include a feedback system to automatically vary the illumination in response to certain changes in the patient's vital signs, including the patient's pulse rate, temperature, and respiratory rate. A patient's vital signs, however, do not provide complete information about the state and status of autonomic nervous system.

U.S. Pat. No. 8,792,986 discloses about an improved device and method for treatment of obesity, especially morbid obesity, other eating related syndromes (e.g., anorexia), and other diseases related to a pathological balance of the autonomic nervous system. The device and method of this invention utilize a sensing element to monitor the patient's heart rate variability and compute the parasympathetic (P1) to parasympathetic (P2) ratio (P1/P2). Based upon the computed ratio, the sensing element then communicates with, and activates as appropriate, at least one gastric electrical stimulation device (GESD) attached to or adjacent to the stomach and/or an intestinal electrical stimulation device (IESD) attached to or adjacent to the small intestine.

In the past, there exist measures for bodily functions such as body temperature, heart rate, lung capacity etc. For example, Heart rate variability (HRV) measurement is commonly used measurement technique to measure regulation of the sinoatrial node, the natural pacemaker of the heart by the sympathetic and parasympathetic branches of the autonomic nervous system. HRV is measured as change in the length of time of consecutive heartbeats. Although such techniques may give a sense of status of autonomic nervous system, they don't effectively help measure or determine the state of ANS (if the brain is in the Parasympathetic or Sympathetic Mode).

Further, traditional neuro-feedback methods and systems that help measure the brain frequencies and use them to determine state of ANS are not very reliable as the outputs are based on the amplitudes (with output being representative of amplitude (uVolts) on the Y Axis and Frequency (bands) along the X axis) and since the output is actually based on the amplitudes, such determination of the state of the ANS are prone to artifacts and noise. One of such method and system is described in U.S. Pat. No. 702423 that discloses a physiological monitoring method and device used for detection of ANS activity in the field of sleep research. The patent discloses about providing output information representative of pulse and slope ratio.

Further the above existed systems and methods referred above do not provide satisfactory response actions to the user to maintain wellness, good mental health and balanced lifestyle.

Thus, the measurement of the homeostasis of users and adequate response actions that can transform lives of the users and help them maintain wellness, good mental health and lifestyle balance in sport, business, teams, marriages etc. is required, therefore there is a need for a novel method, apparatus and neuro-feedback system for processing neuro-signals of a user's brain to derive mean brain frequency that can be used as a measure to study homeostasis conditions of the user's ANS, and provide guided interventions to the user to correct thoughts, perceptions, and stimuli influencing state of the neuro signals or ANS of the user.

### SUMMARY

It is an object of the present invention is to provide a method, apparatus and neuro-feedback system for processing neuro-signals of a user's brain to derive mean brain frequency that correspond to a homeostasis condition of the user's ANS (i.e. sympathetic and/or a parasympathetic condition).

Another objective of the present invention is to derive a Brain Frequency Variability (BFV) index corresponding to a mean brain frequency of the user's brain derived from the neuro-signals or bio signals of the user's brain.

Another objective of the present invention is to provide a neurofeedback system that can be used to train the user's brain using one or more guided interventions that can repair/correct thoughts, perceptions, and stimuli influencing state of mind of the user.

According to preferred embodiment of the present invention, there is provided a neurofeedback system. The neurofeedback system includes a head gear comprising at least one sensor for capturing, in real time, analog neuro-signals from a user's brain, when the user is either at motion or at rest; one or more Analog to Digital Convertors (ADCs) for converting the captured analog neuro-signals to digital neuro-signals; at least one transceiver for transmitting and receiving signals to and from the at least one sensor, and the one or more ADCs; and one or more communication modules in communication with the at least one transceiver for receiving the digital neuro-signals, and in response to receiving the digital neuro-signals transmitting the digital neuro-signals to a neurofeedback program product configured on a computing device communicatively paired with and in communication with the head gear.

According to preferred embodiment, the neurofeedback program product when run on the computing device performing at least one of: eliminating, any interference noise present in the received digital neuro-signals arriving at the computing device using one or more specialized filters, to output a set of frequencies, processing, the outputted set of frequencies to match with a first predefined metric of frequencies classified into a set of standard frequency bands, averaging, the outputted set of frequencies to derive mean brain frequencies, wherein the mean brain frequencies correspond to homeostasis conditions of the user's Autonomic Nervous System (ANS), matching, the mean brain frequencies with a second predefined metric of frequencies classified as Brain Frequency Variability (BFV) Indexes, wherein the BFV index correspond to homeostasis conditions of the user's ANS, displaying, at least one of the mean brain frequencies, and the matching BFV indexes over a Brain Frequency Variability (BFV) Gauge, and in response to the obtained mean brain frequencies and the matching BFV indexes, providing one or more guided interventions to the user to correct thoughts, perceptions, and stimuli that influences state of the analog neuro-signals of the user.

According to preferred embodiment, the analog neuro-signals that are captured from the user's brain correspond to brain's spontaneous electrical activity over a period of time.

According to preferred embodiment, the set of frequencies ranges from 1-44 Hz.

According to preferred embodiment, the one or more specialized filters comprises a Dual Fast Fourier transformation (Dual-FFT) filter.

According to preferred embodiment, the set of standard frequency bands comprising Delta, Theta, Alpha, Beta, and Gamma range of frequency bands.

According to preferred embodiment, the Brain Frequency Variability (BFV) Indexes ranges from 0-10-0.

According to preferred embodiment, the one or more guided interventions provided to the user to correct thoughts, perceptions, and stimuli that influences state of the analog neuro-signals of the user's brain comprises brain training exercises (such as Fluidmynd), breathing exercises, lifestyle pillars, and physical exercises.

Various objects, features, aspects and advantages of the present invention will become more apparent from the following detailed description of the embodiments of the invention, along with the accompanying drawings.

### BRIEF DESCRIPTION OF THE DRAWINGS

The foregoing summary, as well as the following detailed description of preferred embodiments, is better understood when read in conjunction with the appended drawings. There is shown in the drawings example embodiments, however, the application is not limited to the specific system and method disclosed in the drawings.

FIG. 1 illustrates an exemplary process, and a graphical output from a typical prior art neurofeedback system that help measure brain frequencies of a user and use them to determine user's state of ANS;

FIG. 2 illustrates a neurofeedback system of the present invention for receiving, processing neuro-signals of a user's brain to derive brain frequency homeostasis and/or corresponding Brain Frequency Variability (BFV) index as a measure of ANS, according to an embodiment of the present invention;

FIG. 3A-3B illustrates a step by step process from converting analog neuro signals, to discrete time domain digital neuro signals to frequency domain neuro signals, according to an embodiment of the present invention;

FIG. 4 illustrates the neurofeedback system of FIG. 2 that can also be used to train the user's brain using one or more guided interventions, according to an embodiment of the present invention;

FIG. 5 illustrates an exemplary output representation representing user's ANS or homeostasis measured as a function of a set of standard frequency bands along X-axis Vs mean brain frequency and/or BFV indexes along Y Axis, according to an embodiment of the present invention;

FIG. 6A-6B illustrate exemplary representations representing output corresponding to a breathing exercise, where the user's state of ANS represented by the mean brain frequencies and/or the BFV indexes is displayed over a BFV Gauge, according to an embodiment of the present invention;

FIG. 7 illustrates a set of example BFV indexes for a user based on the user's work and schedule for a day, and possible required actions corresponding to the BFV indexes;

FIG. 8 illustrates a method for receiving, processing neuro-signals of the user's brain to derive the brain frequency homeostasis and/or corresponding the BFV indexes as a measure of ANS, according to an embodiment of the present invention;

FIG. 9 illustrates an exemplary user interface containing a set of guided interventions in the form of brain training exercises, breathing exercises, lifestyle pillars, and other physical exercises displayed to the user;

FIG. 10-12 illustrate exemplary user interfaces representing lifestyle pillars exercise, breathing exercise, and brain training exercise in the form of Fluidmynd as displayed on the computing device, according to an embodiment of the present invention;

FIG. 13 illustrates an exemplary user interface that allows changing settings for the breathing exercise of FIG. 10, according to an embodiment of the present invention;

FIG. 14 illustrates an exemplary user interface that allows setting up duration and complexity for exercise sessions, according to an embodiment of the present invention;

FIG. 15 illustrates an exemplary user interface that prompts the user to enter an arbitrary session notes that's saved and sent to cloud storage, according to an embodiment of the present invention;

FIG. 16 illustrates an exemplary report displayed after completion of an exercise session or the report can be retrieved from the cloud storage, according to an embodiment of the present invention;

FIG. 17 illustrates the neurofeedback system of FIG. 2 in use by a group of users for a specific time duration, according to an embodiment of the present invention; and

FIG. 18 illustrates exemplary representation on how the neurofeedback system of FIG. 2 in use by the group of users can be used to testify if the group of users share same state of ANS during a specific time period, according to an embodiment of the present invention.

FIG. 19 illustrates a general block diagram of computing environment or computing device used for the implementation of the proposed invention, according to embodiments of the present invention.

### DETAILED DESCRIPTION

As used in the specification and claims, the singular forms “a”, “an” and “the” may also include plural references. For example, the term “an article” may include a plurality of articles. Those with ordinary skill in the art will appreciate that the elements in the figures are illustrated for simplicity and clarity and are not necessarily drawn to scale. For example, the dimensions of some of the elements in the figures may be exaggerated, relative to other elements, in order to improve the understanding of the present invention. There may be additional components described in the foregoing application that are not depicted on one of the described drawings. In the event such a component is described, but not depicted in a drawing, the absence of such a drawing should not be considered as an omission of such design from the specification.

Before describing the present invention in detail, it should be observed that the present invention utilizes a combination of components, which constitutes a neurofeedback system and associated method thereof. Accordingly, the components have been represented, showing only specific details that are pertinent for an understanding of the present invention so as not to obscure the disclosure with details that will be readily apparent to those with ordinary skill in the art having the benefit of the description herein. As required, the detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

References to “one embodiment”, “an embodiment”, “another embodiment”, “yet another embodiment”, “one example”, “an example”, “another example”, “yet another example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment. The words “comprising”, “having”, “containing”, and “including”, and other forms thereof, are intended to be equivalent in meaning and be open ended in that an item or items following any one of these words is not meant to be an exhaustive listing of such item or items or meant to be limited to only the listed item or items.

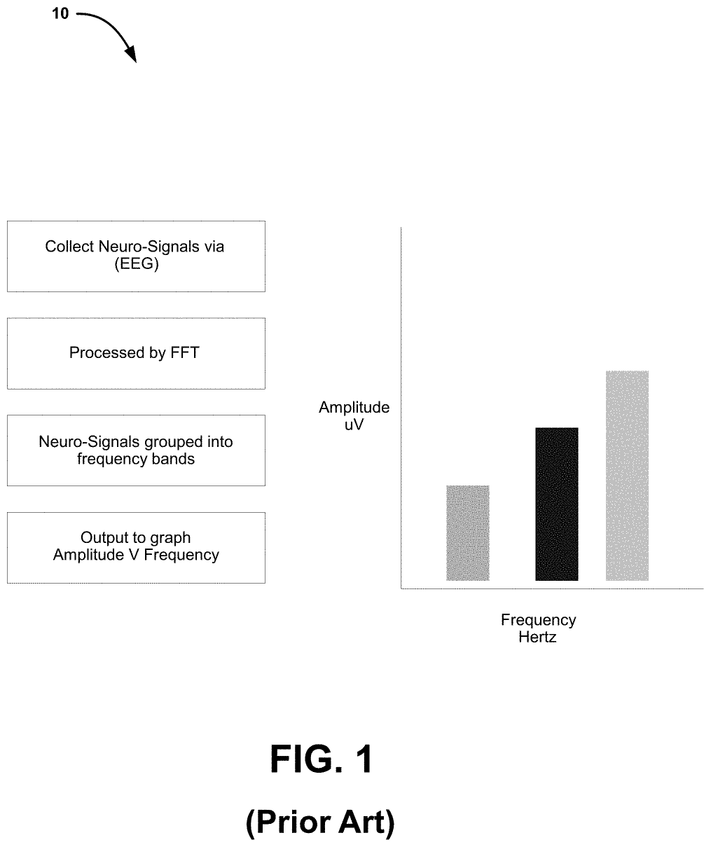

The various features and embodiments of method, apparatus and neuro-feedback system of the present invention will now be described in conjunction with the accompanying figures, namely FIGS. 1-19.

Referring to FIG. 1, an exemplary process, and a graphical output from a typical prior art neurofeedback system that help measure brain frequencies of a user and use them to determine user's state of ANS. As seen, the prior art method 10 includes a step of capturing/collecting analog neuro-signals/bio-signals/EEG signals from a user's brain using a head gear comprising of various sensors. It should be noted, that prior art system discloses about capturing of neuro-signals from the user's brain, when the user is at rest. Further, the method 10 involves a step of conversion of the collected analog neuro-signal to digital neuro-signal for further processing, including applying filters, and then grouping the digital neuro-signals into a set of standard frequency bands (Delta, Theta, Alpha, Beta, and Gamma). Further the method, describes generation of output in some graphical form. As shown, the output graph is represented as a function of standard frequency bands and amplitude. Particularly, as shown in FIG. 1, the graph represents the output in the form of amplitudes (uVolts) along Y-axis Vs standard frequency bands along the X-axis. In such prior art neurofeedback system and method, as the outputs are based on amplitudes, the outputs are generally unstable and unreliable as they are prone to artifacts and noise.

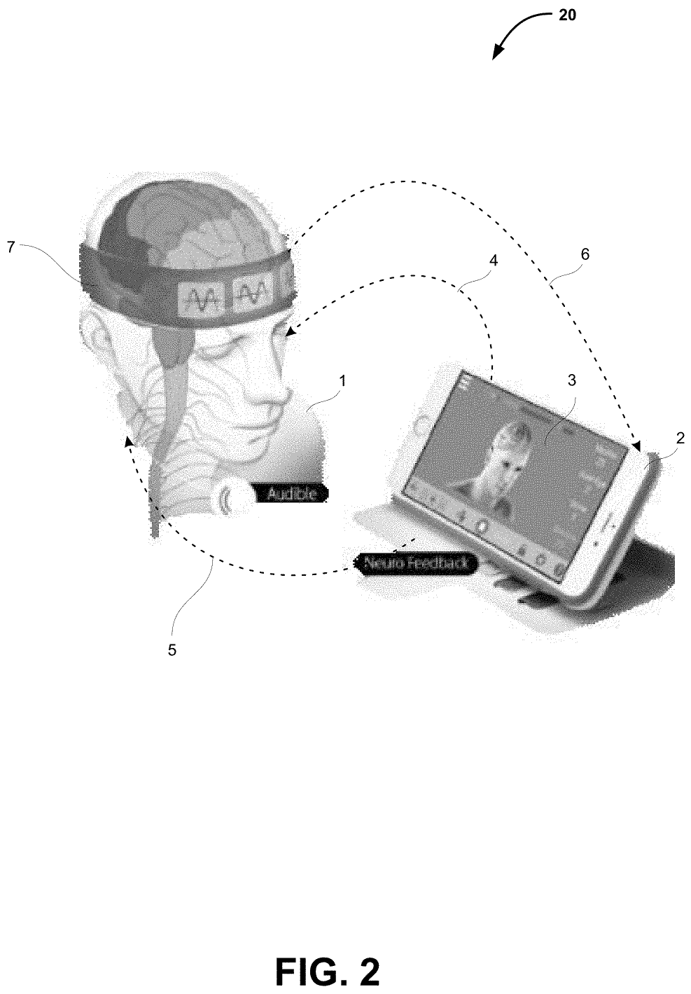

In comparison, as will now be discussed below with reference to FIGS. 2-19, proposed neuro-feedback system/apparatus and method presents output as a function of mean brain frequency and/or corresponding Brain Frequency Variability (BFV) indexes along Y-axis Vs standard frequency bands along the X-axis thereby avoiding any possibility of artifacts or noise in the final output signals.

Referring to FIG. 2, a neurofeedback system 20 of the present invention for receiving, processing neuro-signals of a user's brain to derive brain frequency homeostasis (or mean brain frequency) and/or corresponding Brain Frequency Variability (BFV) index as a measure of ANS is shown, according to an embodiment of the present invention. The proposed neurofeedback system or apparatus 20 includes a head gear or a head wearable accessory 7 equipped with one or more sensors for capturing analog neuro-signals or EEG signals in real time from a user's brain. The user 1 may either be in motion or at rest. The capability of the head gear 7 to capture the analog neuro signals when the user 1 is in motion is described in detail in the Applicant's published pending US20170347905, which is incorporated herein as a reference. The neuro-signals that are captured from the user's brain correspond to brain's spontaneous electrical activity over a period of time. The head gear 7 may include, but are not limited to a headband, a headphone, a wearable gear, an electrode placed on the scalp of the user 1, or any other head wearable accessories. In an embodiment, the head gear 7 may be attached to the scalp of the user 1 using various means, including, but not limited to adhesives, headbands, visors, caps, hats, helmets, clamps, clips, and earring studs. According to a preferred embodiment, the head gear 7 is attached to the head of the user 1 using a headband as shown in FIG. 2. Further, the one or more sensors for capturing the analog neuro-signals or EEG signals in real time from the user's brain may include accelerometers, magnetic sensors, reed relays, RFID, photo-sensors, pressure sensor, piezoelectric sensors, tilt sensors, microphones, radio antennas, proximity sensor, tactile sensor, thermocouples, electro-chemical sensors, gyroscopes, humidity sensors, acoustic sensor, vibration sensors, temperature sensors, hydration sensors, moisture sensor, tilt sensor, inertial measurement unit sensor, compass, inclinometer, altimeter, GPS, pulse-oximeter sensors, EEGs, EKGs, voltmeters, ammeters, capacitance meter, inductance meter, resistance meter, LCR meter, or any combination thereof.

According to the preferred embodiment, the head gear 7 may further include one or more Analog to Digital convertors (ADCs) configured for converting the captured analog neuro-signals to digital neuro-signals.

According to the preferred embodiment, the head gear 7 may further embody one or more communication modules in communication with at least one transceiver configured within the head gear 7 for transmission (indicated by arrow 6) of the digital neuro-signals from the head gear 7 to a neurofeedback program product or an application program product configured on a computing device 2 paired with and in communication with the head gear 7. The pairing of the computing device 2 with the head gear 7 may be via wireless or wired networks known in the art. For example, wife or Bluetooth or Internet. The transmission of the digital neuro-signals from the head gear 7 to the program product configured on the computing device 2 is via network, preferably but not limited to Internet/WiFi or Bluetooth.

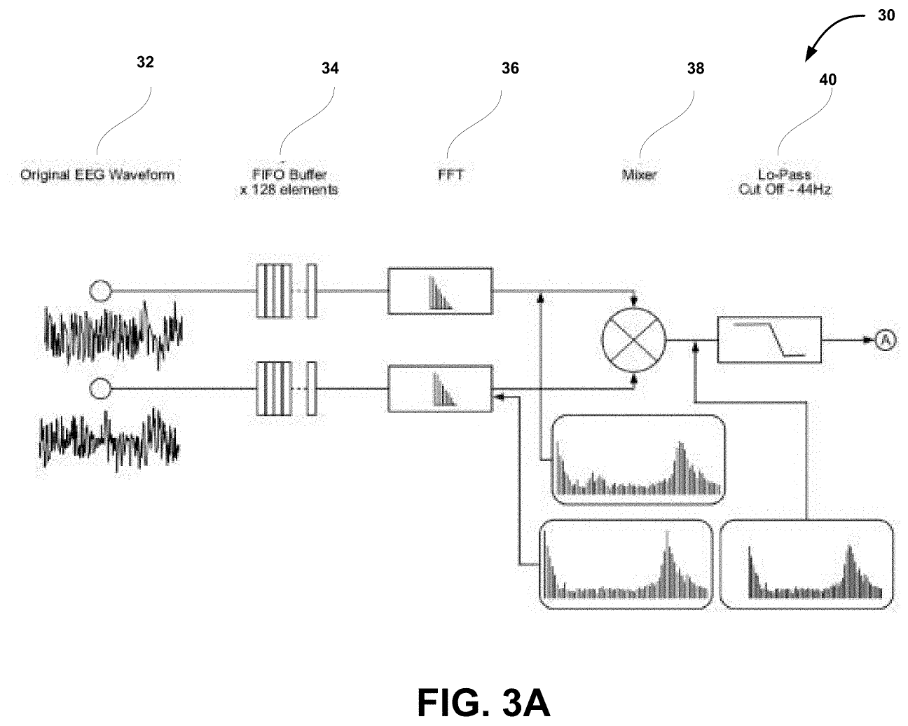

According to the preferred embodiment, the neurofeedback program product configured on the computing device 2 upon receiving the digital neuro-signals, runs one or more specialized filters, particularly Dual parallel Fast Fourier transformation (Dual-FFT) on the received digital neuro-signals to eliminate any interference noise that may be present in the received digital neuro-signals, and output a set of frequencies ranging from 1-44 Hz. The process as to how the Dual parallel FFT is implemented onto the digital time domain neuro-signals obtained from the user's brain via the head gear 7 is described in detail in with respect to FIG. 3A-3B below.

Referring to FIGS. 3A-3B, as seen at step 32, the analog neuro signals from the user's brain are initially captured by the sensors present in the head gear 7, which is then possessed by the ADCs to generate digital neuro signals which are then transmitted to the computing device 2 via the communication module. At step 34, the computing device 2 embodying the neurofeedback program product receives the digital neuro-signals and as shown, at step 34, the computing device 2 store the digital analog signal (available in discrete form) in a FIFO (first in first out) buffer on a first-in, first-out basis. The FIFO buffers are used to manage asynchronous discrete digital signals/data at different time. The storage structure of FIFO buffer is typically an array of contiguous memory. The discrete digital data/signal is written to the “head” of the buffer and read from the “tail”. When the head or tail reaches the end of the memory array, it wraps around to the beginning. If the tail runs in to the head, the buffer is empty.

At step 36, the computing device 2 is configured to process the received digital discrete neuro signals/data using dual parallel fast Fourier transformation (FFT) mechanism. The FFT is a fast implementation of the Discrete Fourier Transform (DFT) wherein the received digital discrete signals/data are converted from time domain digital discrete signals/data to corresponding frequency domain neuro signals/data. The dual parallel fast Fourier transformation (FFT) mechanism use equation (3), this results by performing FFT on equation (1) and equation (2), to convert time domain digital discrete signals/data to corresponding frequency domain neuro-signals/data.

$$\begin{matrix}{X}_{k} = {\sum }_{n = 0}^{N - 1} {x}_{n} \cdot {e}^{- i 2 π kn / N} & ( 1 ) \\ {X}_{k} = {\sum }_{n = 0}^{N - 1} {x}_{n} \cdot {e}^{- i 2 π kn / N} & ( 2 )\end{matrix}$$

As shown above, the multidimensional DFT transforms an array xₙ with a d-dimensional vector of indices n=(n₁, n₂, . . . n_{d}) by a set of d nested summations (over nⱼ=0 . . . Nⱼ₋₁ for each j), where the division n/N, defined as n/N=(n₁/N₁, n₂/N₂, . . . n_{d}/N_{d}), is performed element-wise. Further, performing dual parallel FFT on the equation (1) and the equation (2), results equation (3)

$$\frac{N}{{N}_{1}} O ( {N}_{1} log {N}_{1} ) + … + \frac{N}{{N}_{d}} O ( {N}_{d} log {N}_{d} ) = O ( N [ log {N}_{1} + … + log {N}_{d} ] ) = O ( N log N ) . \qquad ( 3 )$$

At step 38, the frequency domain neuro signals/data are inputted to a mixer. The mixer shift signals from one frequency range to another. For example, consider a scenario where two signals at frequencies f1 and f2 are applied to a mixer along with the signal from a local oscillator, the mixer produces new signals as the sum f1+f2 and difference f1−f2 of the original frequencies. The mixer produces a frequency shifted neuro signals/data from the inputted frequency domain neuro signals/data. Then, the output signal of mixer is inputted to a low pass filter.

At step 40, the low pass filter having a cutoff frequency, for example 44 Hz, passes the neuro signals with a frequency lower than the cutoff frequency and attenuates the neuro signals with a frequency higher than the cutoff frequency. The output of the low pass filter (denoted as “A”) is inputted to a moving averages filter.

At step 42, the moving averages filter use equation (4) while considering a slew rate (for example, 2πfv) for smoothing an array of sampled data (neuro data from the low pass filter). The moving average filter is a simple Low Pass FIR (Finite Impulse Response) filter commonly used for smoothing an array of sampled data. In an example, the moving average filter takes M samples of input at a time and performs the average of those M-samples and produces a single output point. The moving average filter eliminates unwanted noisy component (e.g., movement artifact) from the intended data (neuro signals).

$$MA ( v ) := {\sum }_{x ∈ V} {p}_{v} ( x ) \cdot f ( x ) \qquad ( 4 )$$

At step 44, the EEG/neuro data from the moving average filter is compared with the data of Table 1, to determine the extent of concussion based on the EEG data. Further, the computing device 2 is configured to cause to display data related to the extent of concussion.

Turning back to the description outlined for FIG. 2, upon outputting the frequencies ranging from 1-44 Hz, the neurofeedback program product further processes the outputted frequencies 1-44 Hz to match with a first predefined metric of frequencies classified into a set of standard frequency bands (Delta, Theta, Alpha, Beta, and Gamma). The Table 1 below shows classification of the outputted frequencies form the Dual parallel FFT into a set of standard frequency bands.

TABLE 1

| Name | Frequency range (Hz) | User State of mind |
|---|---|---|
| Delta | 0-4 | Sleep, unconscious processing |
| Theta | 4-7 or 4-8 | Deeply relaxed, inwardly focused |
| Alpha | 8-12 or 8-13 | Very relaxed, passive attention |
| Beta | >13 | External attention |
| SMR Beta | 12-15 | Relaxed, external attention |
| Low Beta | 15-18 | Active, external attention |
| Beta | 18-32 | Active, external attention |
| High Beta | 32-38 | Anxiety, external attention |
| Gamma | >38 | Peak performance states or a |
| | | consolidation frequency |

Although, Table 1 shown above could be helpful in a substantial way to give an overall assessment of user's state of mind or brain, still it is desired to further process the outputted frequencies by Dual Parallel FFT (0-44 Hz) to assess state of the user's ANS or mind. As shown in example Table 1, if the outputted frequency of the user 1 is 3 Hz, then the frequency is classified as Delta and the computing device 2 may determine user's state of brain/mind as unconscious. Similarly, if the frequency determined by Dual parallel FFT as 20 Hz, then the computing device 2 may determine user's state of brain or mind as an anxiety state. Even though classifying frequency outputted by Dual Parallel FFT provides an average knowledge about the user's state of mind. It may not give indication of homeostasis conditions of the user's ANS, particularly a sympathetic condition and/or a parasympathetic condition.

Thus, in order to assess homeostasis conditions of the user's ANS, once the neurofeedback program product has processed the outputted frequencies 1-44 Hz to match with the first predefined metric of frequencies as shown in Table 1, the program product then further averages the outputted frequencies obtained from the Dual FFT filter to obtain mean brain frequency (or also termed as “Brain Frequency Homeostasis (BFH)”).

The mean brain frequencies or BFH can be mathematically derived as the sum of the product of the spectrogram intensity (intensity of spectrum of frequencies obtained from Dual parallel FFT filter) and the spectrogram frequency, divided by the total sum of spectrogram intensity.

$${f}_{mean} = \frac{{\sum }_{i = 0}^{n} {I}_{i} \cdot {f}_{i}}{{\sum }_{i = 0}^{n} {I}_{i}}$$

Where fₘₑₐₙ=Mean Frequency
n=Number of frequency bins in the spectrum
fᵢ=Frequency of spectrum at bin i of n
Iᵢ=Intensity of spectrum at bin i of n

Once the mean brain frequency or BFH is obtained by processing the outputted frequencies from the Dual parallel FFT, the neurofeedback program product then matches the obtained mean brain frequency or BFH with a second predefined metric of frequencies classified as Brain Frequency Variability (BFV) indexes as shown in Table 2 below, where BFV indexes correspond to homeostasis conditions of the user's ANS.

TABLE 2

| Mean Brain | | Pointer Angle of |
|---|---|---|
| Frequency Or BFH | BFA Indexes | Gauge (degrees) |
| 1.00 | 1 | 210 |
| 2.00 | 2 | 201.25 |
| 3.00 | 3 | 192.25 |
| 4.00 | 4 | 183.75 |
| 5.00 | 5 | 175 |
| 6.00 | 6 | 158.33 |
| 7.00 | 7 | 141.66 |
| 8.00 | 8 | 125 |
| 9.00 | 9 | 107.5 |
| 10.00 | 10 | 90 |
| 11.00 | 10 | 90 |
| 12.00 | 9 | 72.25 |
| 13.00 | 8 | 63.75 |
| 14.00 | 7 | 55 |
| 15.00 | 6 | 46.66 |
| 16.00 | 6 | 38.33 |
| 17.00 | 5 | 29.99 |
| 18.00 | 5 | 21.66 |
| 19.00 | 4 | 13.33 |
| 20.00 | 4 | 5 |
| 21.00 | 3 | 1.5 |
| 22.00 | 3 | 358 |
| 23.00 | 2 | 354.5 |
| 24.00 | 2 | 351 |
| 25.00 | 2 | 347.5 |
| 26.00 | 1 | 344 |
| 27.00 | 1 | 340.5 |
| 28.00 | 1 | 337 |
| 29.00 | 0 | 333.5 |
| 30.00 | 0 | 330 |

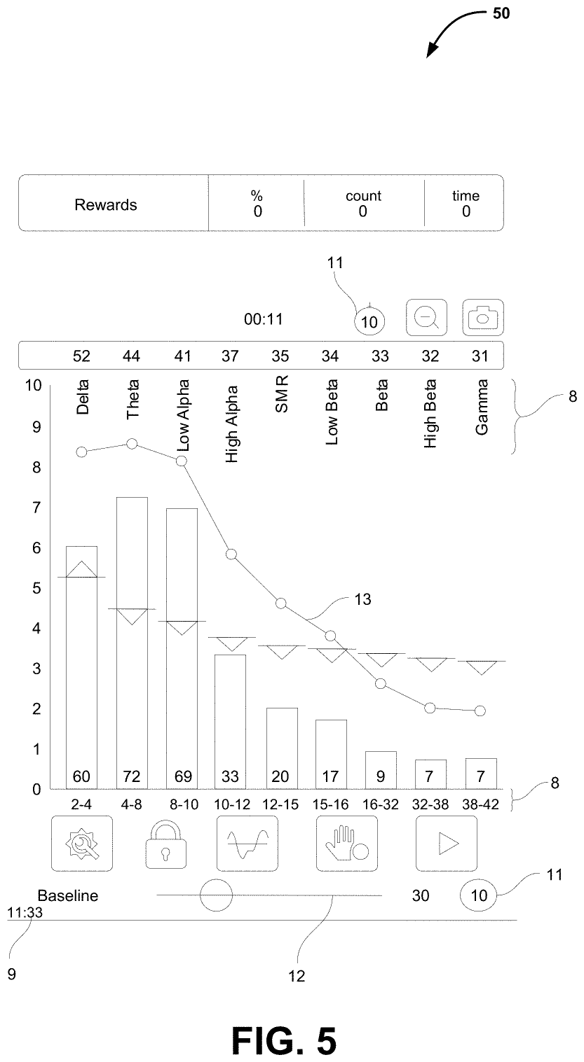

Referring to Table 2 above shows mean brain frequency for the user in first column, and BFV indexes corresponding to mean brain frequencies in second column. Further, a third column of Table 2 shows Gauge pointer deflection corresponding to each of BFV indexes as shown in FIG. 6A-6B. For example, when the mean brain frequency estimated by the neurofeedback program product is 6 Hz then the corresponding BFV index is also 6. Similarly, when the mean brain frequency estimated by the neurofeedback program product is 20 Hz, the corresponding BFV index is 4 and so on. It can be noted from the above table that, the mean brain frequencies of 10 Hz, 11 Hz have an ideal BFV index value as 10 that represents homeostasis condition of ANS. The BFV index 10 denotes that the bodily functions of the user's ANS is balanced. The mean brain frequency lower than 10 Hz and greater than 11 Hz shows fluctuation in the BFV index, still, the mean brain frequency of 7-9 Hz with corresponding BFV indexes as 7-9, respectively, and the brain mean frequency 12-14 with corresponding BFV indexes 9-7, respectively could be considered practically acceptable and the state of ANS could be in either in the sympathetic or the parasympathetic mode based on the work the user 1 is involved in. Very high brain mean frequency (say 25-30 Hz for example) and very low mean brain frequency (1 or 2 Hz for example) represent critical condition of user's ANS.

As derived herein, the BFV indexes ranges from 0 to 10 to 0 (0-10-0). The BFV index of 10 represents an ideal condition where the user 1 is at optimum homeostasis condition, meaning the user's bodily functions are all balanced or regulated properly, or is in its equilibrium state. Any BFV index below 10 means the user's ANS state is not in an equilibrium state/balanced state and typically means that the user 1 is either in the sympathetic and/or the parasympathetic condition.

The sympathetic mode relates to “Fight or Flight” system. It activates the body and mind of the user 1 for exercise and work and it prepares the body to meet real or imagined threats to its survival. For example, people in office working all day usually have this state of mind. Similarly, the parasympathetic mode is known as “Rest & Digest”. When the parasympathetic mode is activated, we generally tend to relax, calm and slow down. For example, when the person is sleeping or taking a nap.

Typically, when the BFV indexes don't fluctuate much with respect to the ideal BFV index 10, say the BFV index just changes to index values 8 or 9, then ideally it would mean the user's ANS is not in homeostasis state, however, indexes 8 or 9 would denote practically acceptable condition in either of the parasympathetic mode or the sympathetic mode and no corrective actions to correct thoughts, perceptions, and stimuli that influences state of the user's brain or analog neuro signals is typically needed while considering such indexes. When the BFV indexes fall further down say to 4 or 5 or even lower, then it can be estimated that the user 1 is undergoing critical problems, may it be for example, stress level is high, or workload is heavy at workplace, or is having anxiety or feeling too much laziness, and so on and corrective actions to correct user's thoughts, perceptions, and stimuli might be required.

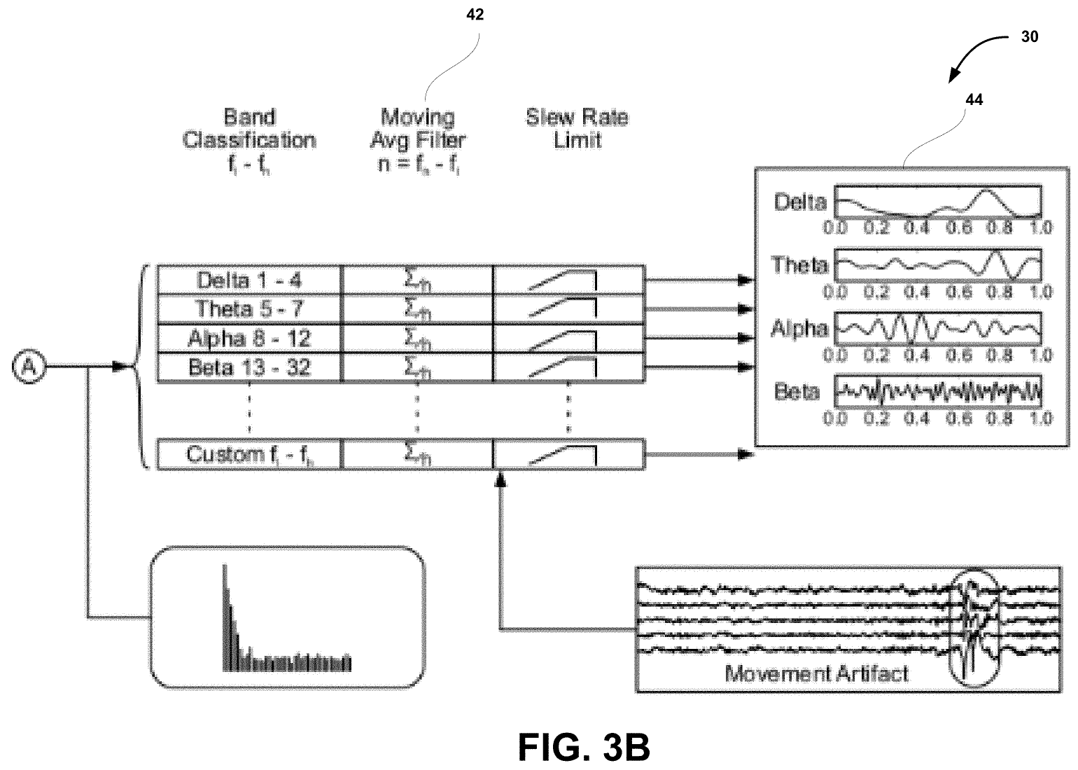

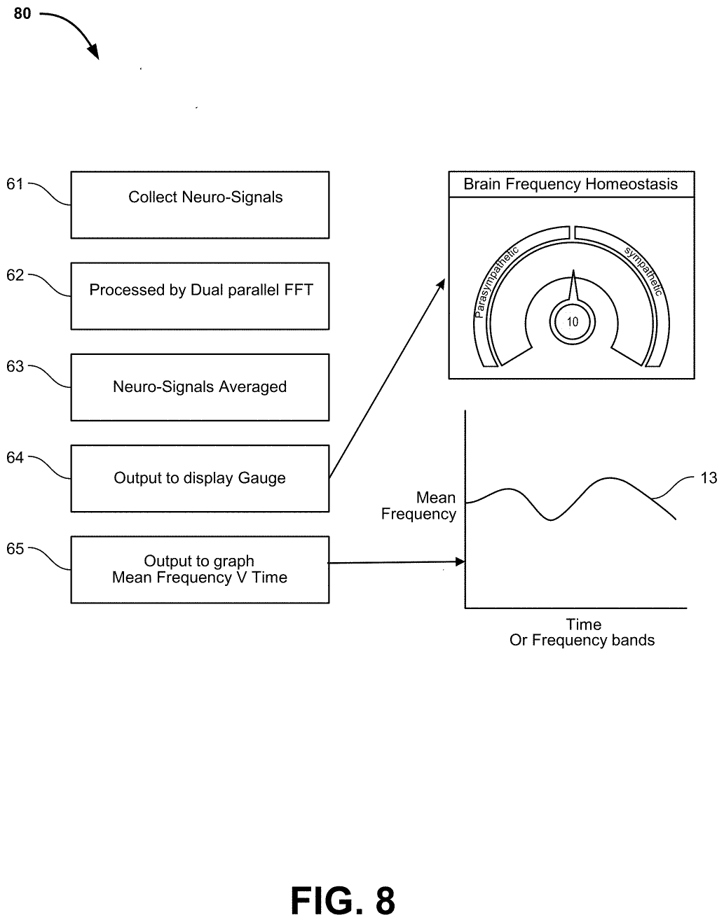

Based on the state of work the user 1 is involved in and the mean brain frequency or BFV index value one can know easily the state of the user's ANS and accordingly guided interventions 31 or corrective actions can be advised to or take up by the user 1 as shown in FIG. 4. The guided interventions 31 provided to the user 1 to correct thoughts, perceptions, and stimuli that influences state of the analog neuro-signals of the user's brain. The guided interventions 31 may be in the form of brain training exercises, breathing exercises, lifestyle pillars, and other physical exercises 32 as shown in FIG. 4. The user 1 can take up one or more of these brain training exercises, breathing exercises, lifestyle pillars, and other physical exercises 32 over the computing device 2 over the neurofeedback program product configured thereon. In general, these exercises may be in the form of visuals, for example in the form of avatar 3 or audio as indicated by arrows 4 and 5 in FIG. 2. Some example situations, that might require the guided interventions 31 to be given or taken up by the user 1, say, if the user 1 is undergoing some critical financial problem and is unable to take sleep properly, this may result in lower BFV index value say 4 or 5 or 6 and since the user 1 is in the relaxing state (no matter if he is unable to take sleep), it could be estimated that the user's ANS is in the parasympathetic mode. Likewise, if the user 1 starts his job he may have the BFV index close to ideal BFV index 10. However, suddenly, if the user is assigned a few new assignments and is asked to complete them in next few hours, it is likely the user's ANS would fluctuate due to stress and the BFV index may fall to 4 or 5 or 6 based on the user's caliber. One can easily estimate state of the user's ANS, which happens to be non-homeostasis, sympathetic mode. A more detailed outlook on the different types of guided interventions taken up or provided to the user to correct thoughts, perceptions, and stimuli that influences state of the user's brain will be discussed below with respect to FIGS. 9-16.

Further, as shown and described above, once the neurofeedback program product of the computing device 2 matches the obtained mean brain frequency with corresponding BFV indexes during the exercises (say breathing exercise), the program product then displays the mean brain frequency and corresponding BFV over a Brain Frequency Variability (BFV) Gauge as shown in FIGS. 6A-6B. FIGS. 6A-6B shows exemplary representations 60 of output displayed over the computing device 2 in the form of BFV Gauge 13 (during breathing exercise for example), particularly the BFV indexes (ranging from 0-10-0) and corresponding to mean brain frequencies of the user 1 is displayed. The gauge 13 is preferably shown to have two sections (one onto the left indicative of the parasympathetic mode of the ANS, and one onto the right indicative of the sympathetic mode of the ANS), with a centrally located pointer 14 rotating around those two sections and pointing to a specific BFV index 0-10-0. As seen in the FIG. 6A, the pointer 14 of the gauge 13 points to the center of the meeting point of the two sections, and is ideally the homeostasis condition and represents BFV index as 10. Any BFV indexes (0-9) pointed by pointer 14 of the gauge 13 onto the right relates to the sympathetic mode of the ANS, For example the FIG. 6B, shows the symphonic mode of the ANS with BFV value 9 (corresponding to BFH 11.62 Hz as shown circled).

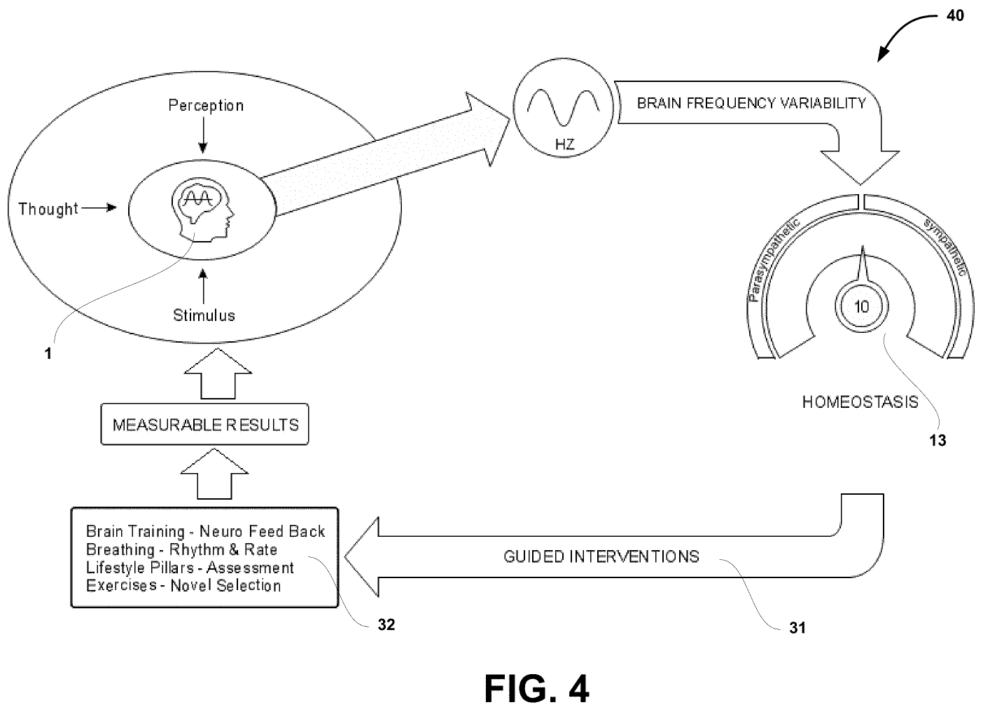

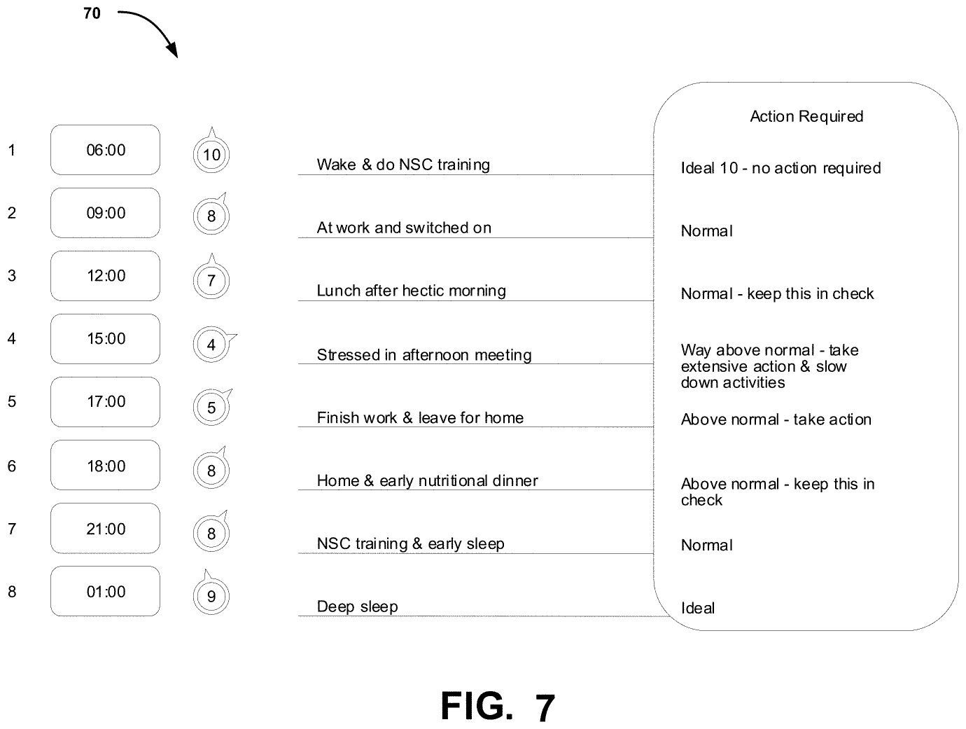

Besides representing the BFV index and corresponding mean brain frequency using the gauge 13, the program product is able to represent the output in more detailed form as well, for example, as shown in FIG. 5, a combined graph 50 is shown, where the baseline 12 indicates the mean brain frequencies (0-30 Hz) obtained after averaging the frequencies outputted by the Dual FFT filters, the mean brain frequency 11.33 Hz and corresponding BFV index value 10 is represented by 9 and 11, respectively. The graphs as represented in FIG. 5 and FIG. 8 (onto the bottom right of the FIG. 8), mainly shows output as a function of mean brain frequency (graph line 13) and/or corresponding BFV indexes along Y-axis Vs standard frequency bands/time along the X-axis. Since the output is completely a function of Frequency Vs Frequency there is negligible chances of any noise or artifacts to affect the output.

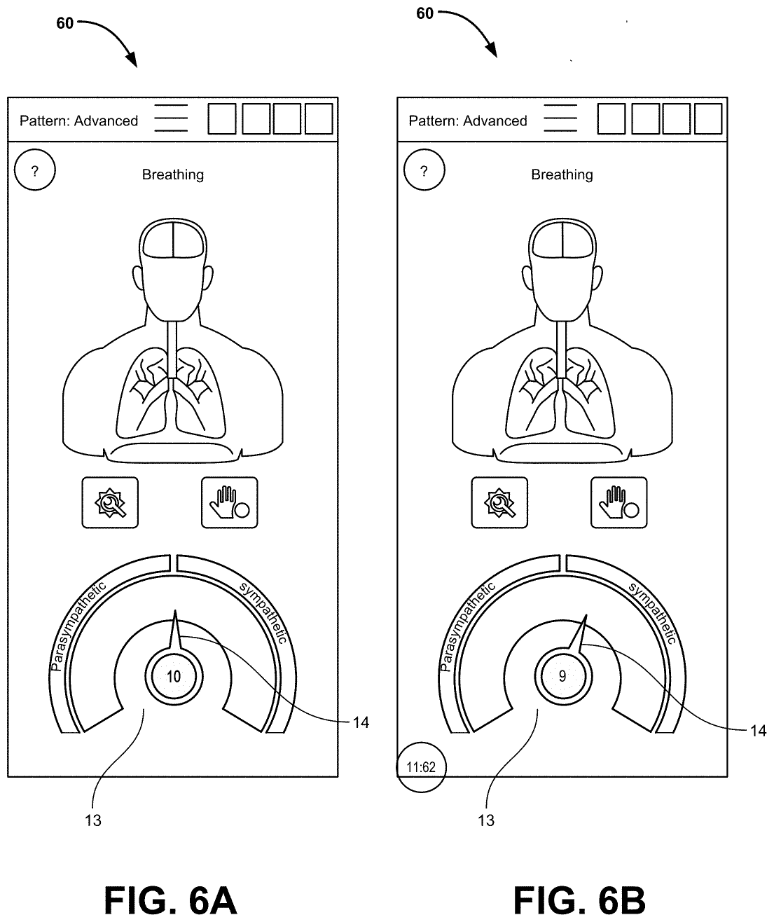

Referring now to the example 70 represented in FIG. 7, a set of example BFV indexes for a user based on the user's work and schedule for a day, and possible required actions corresponding to the BFV indexes is shown. As seen, the user's specific day's schedule shows BFV indexes fluctuating from 4 to 10. In the morning, when the user 1 wakes up from deep sleep at 6 AM, the measure of his/her ANS usually shows BFV index as 10 (an ideal condition) so no corrective actions/feedback or the guided interventions are needed for the user 1, as his body is in balanced state already. Later, at 9 AM, when the user 1 is at work and starts working, the measure of his/her ANS changes to 8 (i.e, BFV index=8), which is practically acceptable BFV index and again no corrective actions/feedback or the guided interventions may be needed. As the day progresses, at 12 PM, after lunch when the user 1 has a hectic work load, the user's state of the ANS then shows sympathetic mode with BFV index as 7. Even this state can be regarded as normal and no corrective actions/feedback or the guided interventions may be needed, however, the user 1 may be advised to keep a watch/check. Next, at 3 PM, the measure of the user's ANS changes to 4 (i.e, BFV index=4), as by this time the user 1 may be stressed due to the work (may be back to back meetings for example), and the user 1 thus may be advised to take corrective actions or can take up some guided interventions and slow down the work. Similarly, by 5 PM, the user 1 may be preparing to go back home from work, and then thus be a little relaxed but still the BFV index may measure as 5, which is again way lesser than the ideal BFV value and one should take appropriate corrective actions or guided interventions. By 6 PM or 9 PM, when the user reaches home, he will have nutritional dinner and could relax and go for early sleep, and thus the BFV index will slowly raise, say BFV=8, as shown which is practically nearing the idea homeostasis BFV index 10/nearing normal ANS. Later, at 1 AM midnight, the user 1 will be fully calmed or relaxed or be in deep sleep, and the BFV index will raise further to 9 or 10 by the time when the user wakes up again for next day. Thus, using the proposed neurofeedback system, the user can be trained to attain the ideal BFV index of 10 which correlates to a balanced Homeostasis providing various measures/control actions or guided interventions (either via audio or video or physical exercises form), whenever, BFV index of the user's ANS lowers down.

Referring to FIG. 8 in conjunction with FIG. 2 (described above), a method 80 for receiving, processing neuro-signals of the user's brain to derive the brain frequency homeostasis or mean brain frequency and/or corresponding the BFV indexes as a measure of ANS is shown. At step 61, broadly capturing, in real time, analog neuro-signals from a user's brain is described. The captured analog neuro signals from the user is first converted to digital neuro-signals before the neuro signals are passed (via communication module) to a neurofeedback program product configured or installed on a computing device for further processing.

At step 62, processing of the received digital neuro signals is described. The neurofeedback program product runs one or more specialized filters to eliminating, any interference noise present in the received digital neuro-signals arriving at the computing device in order to output a set of frequencies (ranging from 1-44 Hz).

At step 63, averaging of the outputted set of frequencies to derive mean brain frequencies that correspond to homeostasis conditions of the user's ANS is described. Optionally, the derived mean brain frequencies can be matched with a predefined metric of frequencies classified as BFV Indexes Like mean brain frequencies, the BFV index also correspond to homeostasis conditions of the user's ANS.

At step 64, displaying of BFV indexes and optionally at least one of the mean brain frequencies, over a Brain Frequency Variability (BFV) Gauge is described. At step 65, display of output as a function of mean brain frequency (graph line 13) and/or corresponding BFV indexes along Y-axis Vs. standard frequency bands/time along the X-axis is shown.

Referring to FIG. 9, an exemplary user interface 90 containing a set of guided interventions in the form of brain training exercises, breathing exercises, lifestyle pillars, and other physical exercises as displayed to the user is shown. As described, once the mean brain frequency or the BFV indexes corresponding to state of user's ANS is derived and analyzed, the user can then go for one or more guided interventions over the computing device via neurofeedback program product, particularly lifestyle pillars, breathing exercises, Fluidmynd exercise and other brain training exercises, and other interventions 32 that can intervene with the thoughts, perceptions, and stimuli to repair state of the user's neuro-signals (or in another word user's ANS). This is achieved, either by uplifting or decreasing the mean brain frequency or the BFV indexes.

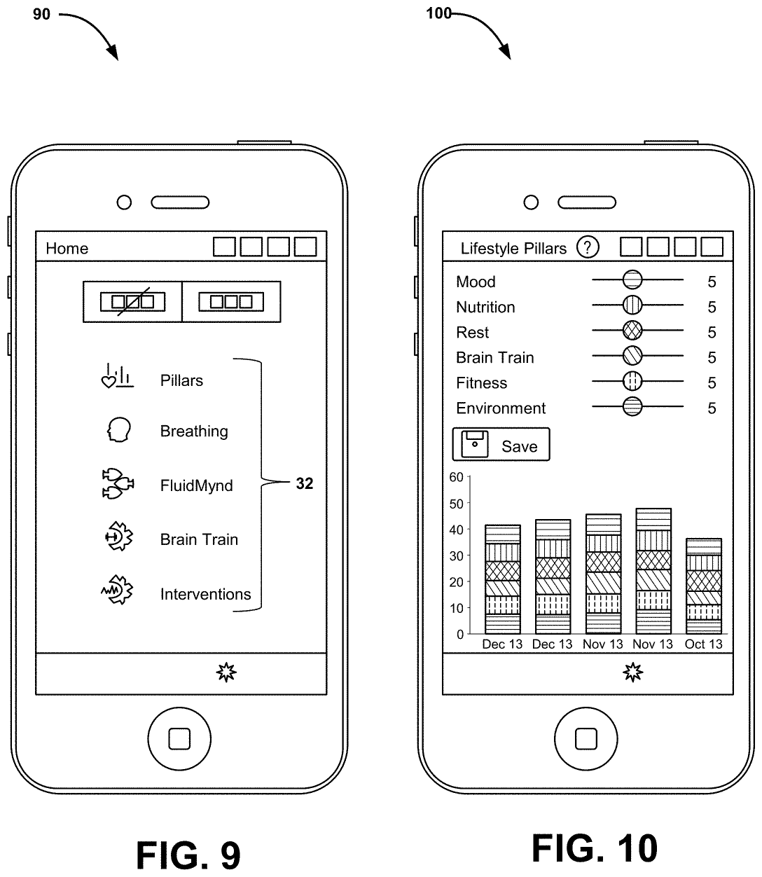

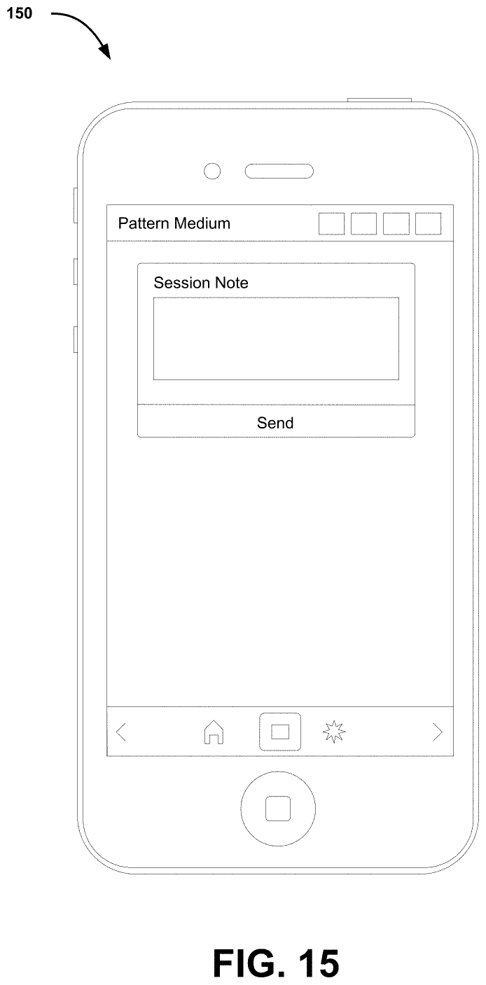

Referring to FIG. 10, an exemplary user interface 100 representing lifestyle pillars as displayed on the computing device is shown, according to an embodiment of the present invention. This type of intervention is manual intervention, wherein once the user selects the lifestyle pillars as choice for the guided interventions, the user is prompted to rate a set of attributes related to his lifestyle. In an example, the set of attributes includes mood, nutrition, rest, brain train, fitness, and environment. Although, there are six attributes listed and used for the purpose of this invention, there can be lesser or more number of attributes used. In the example shown, the user is prompted to rate each of these attributes in a scale of 10. As seen in the example, the user has rated each of these attributes as 5. The user may use the lifestyle pillars as a guided intervention to correct his perception, thought and stimuli to maintain balanced homeostasis, and enter values against all the attributes, and save them along with any possible session note (such as one shown in the FIG. 15) which can then be transmitted to cloud storage for later retrieval or report generation. The user or instructor/trainer of the user can always analyze the report after each session or collective report (in the form of bar graph) for specific time interval such as a month as shown in FIG. 10. As seen, the monthly graph shows, the collective attribute values along Y axis and months along X axis.

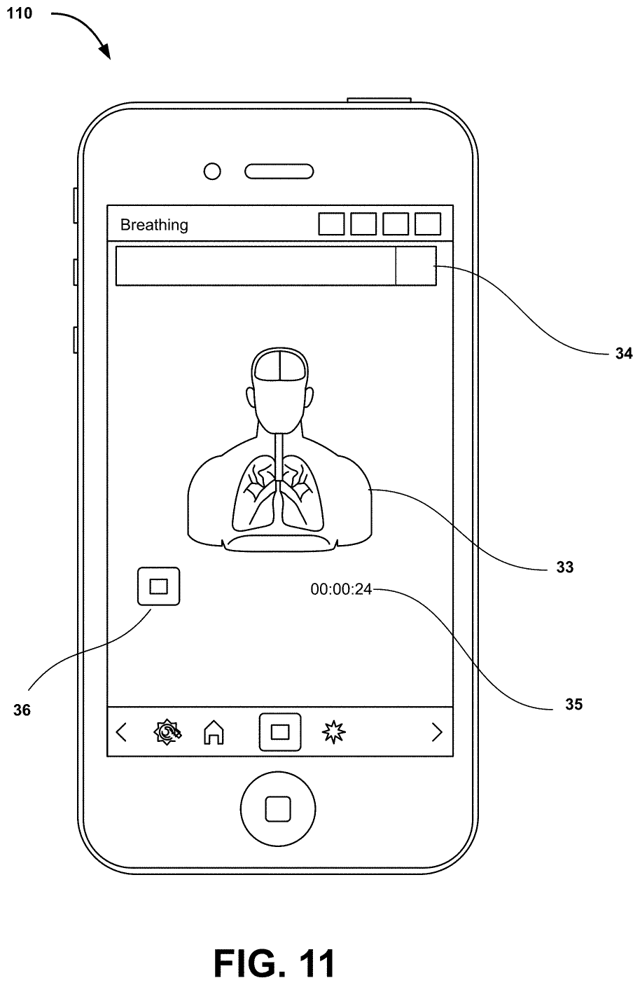

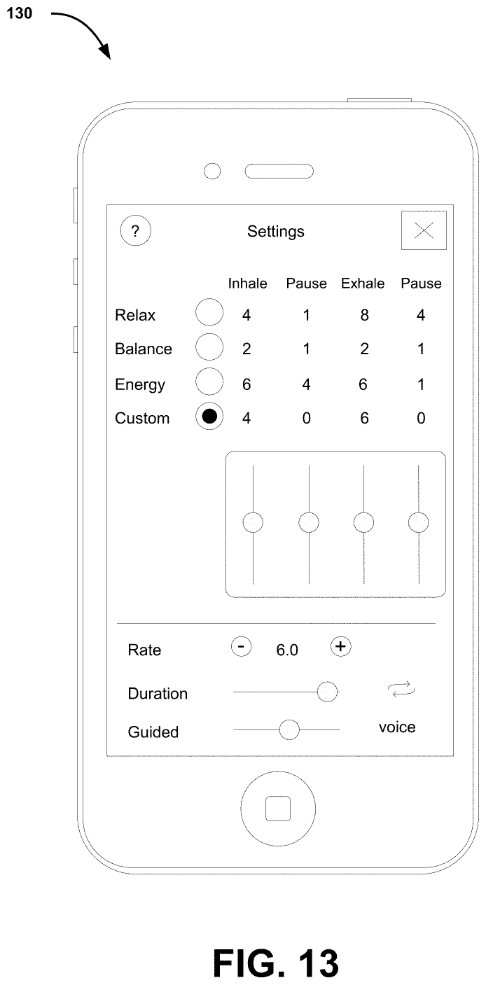

Referring to FIG. 11, an exemplary user interfaces 110 representing breathing exercise as displayed on the computing device is shown, according to an embodiment of the present invention. As seen, the interface 110 shows, a breath animation 33, a trend bar 34, a time lapsed 35, and a start/stop session button 36. The start/stop session button 36 can be used by the user or trainer to initiate or stop the session. The breath animation 33 would show the brain and lungs in animation form, where the brain and lungs may be turned green or red based on breathing rhythm and rate. The trend bar 34 would show BFV index level. A vertical line within the bar 34 appears by tapping the bar 34 to show the value of the BFV index. The time lapsed 35 indicator will show how long the session was run. The user can wear the head gear and either he himself (or his trainer) run this guided intervention over the computing device to correct any identified deficiency in the user (measured in terms of BFV index or mean brain frequency). Further, the user or the trainer if required can customize settings for the breathing exercise using an exemplary graphical user interface 130 shown in FIG. 13. As seen, the user interface 130 allows to set how many times the user should inhale and exhale and how many times the user can pause after inhale and exhale. In the example, inhale:pause:exhale:pause ratio is shown as 4:0:6:0. The user can also set the rate at which breathing should be performed, in the example shown, it is shown as 6. The user can further set the duration for breathing exercise. Also, the user can (if desired) set the breathing exercise session as voice guided session/unguided session.

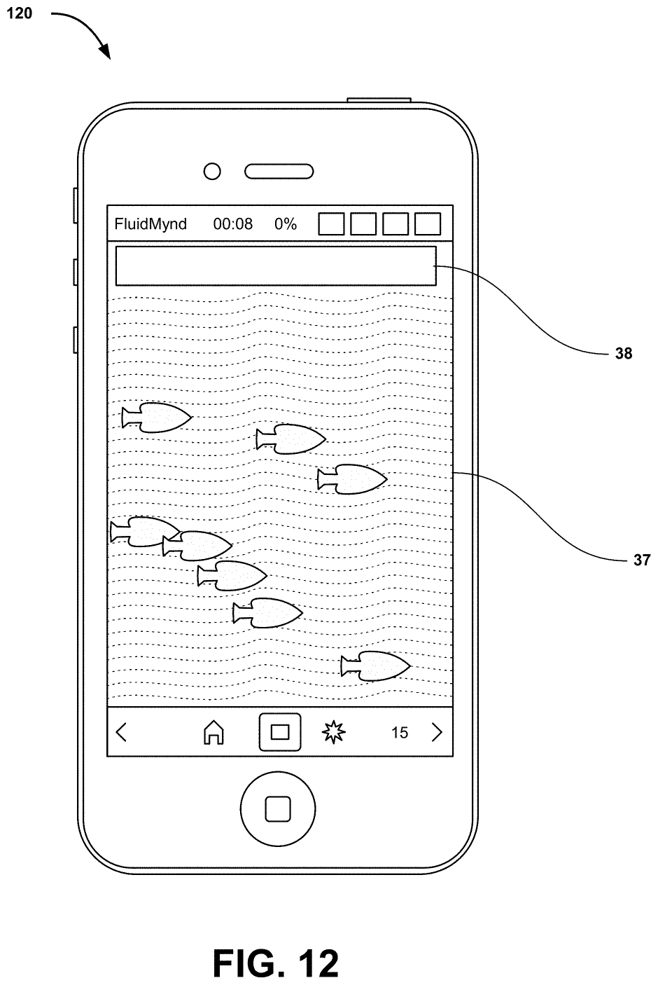

Referring to FIG. 12, an exemplary user interfaces 120 representing brain training exercise in the form of Fluidmynd game as displayed on the computing device is shown, according to an embodiment of the present invention. As seen, the interface 120 shows a tank 37 section holding water and where fishes swim, and a trend bar 38. When the user selects this form of guided intervention to perform exercise, and if the fishes turn blue in color and swim slowly at the bottom of the tank 37 then that would indicate the user has a calm mind or balanced state of ANS. If the user's ANS is unbalanced or the user has some anxiety or under stress then the fishes would turn orange and dart up to the top of the tank. The trend bar 38 would indicate BFV index level during the exercise.

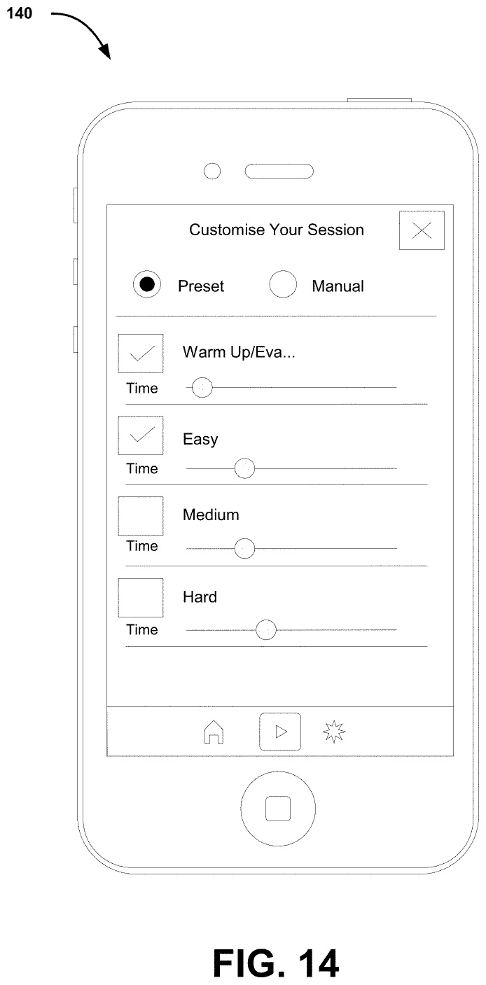

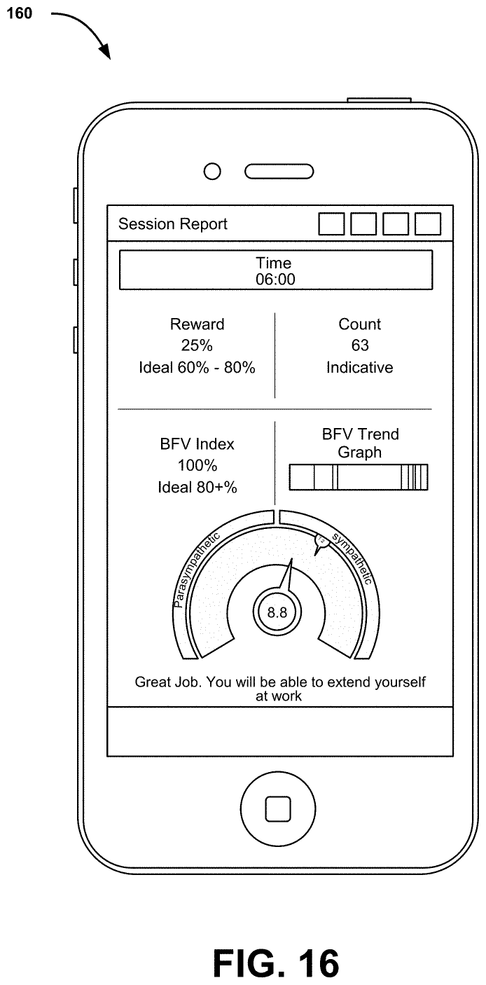

Referring to FIG. 14, an exemplary user interface 140 that allows setting up duration and complexity for exercise sessions, according to an embodiment of the present invention. The user interface 140 may be used to preset or manually set training sessions for breathing exercise, or brain training exercise. Using this user interface 140 user can set the timer for the Session. When the session times out a popup would then appear which would prompt the user to enter arbitrary session notes (as shown in FIG. 15), once note is entered and a send button is presses note is sent to the cloud for storage and a session report is displayed instantly or may be later retrieved. FIG. 16 in particular illustrates an exemplary report displayed after completion of an exercise session, according to an embodiment of the present invention. The report shown is for the breathing exercise. As shown, the report shows the duration for which the exercise session was run. In the example, it is shown as 6 mins. The BFV is represented to show a value 8.8 over the BFV gauge using a pointer. The value of 8.8 is close to ideal value and that shows the user's state of ANS (sympathetic mode) is pretty good. Further BFV is also represented in terms of % and trend graph. As seen, the BFV % shows 100% (wherein the ideal value is supposed to be 80+%), so the state of user's ANS is determined to be good. The report further shows rewards user obtained after the exercise session, which is shown as 25%. The report also shows the breath counts.

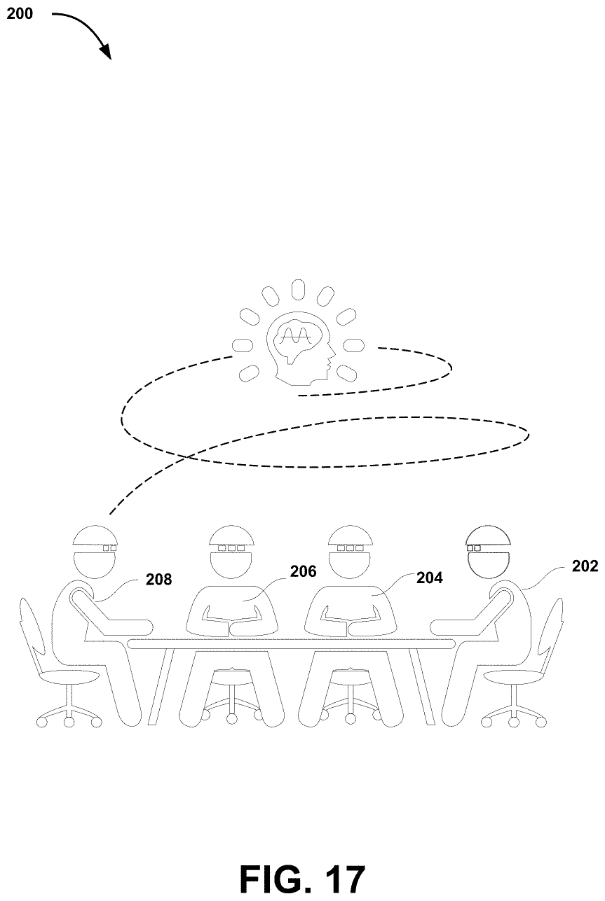

Referring to FIG. 17, the neurofeedback system of FIG. 2 in use by a group of users (such as users 202-208) for a specific time duration is shown, according to an embodiment of the present invention. During the exercise, the group of users (such as users 202-208) wear their respective head gears and undergo neurofeedback training, and during training their mean brain frequencies can then be compared to testify if the group of users share same state of ANS or not (if they share the same mean brain frequency). The use of this group flow or estimation of state of ANS within a group of users can be implemented in various application areas such as but not limited to organizations to boost their business, educational industry such as college and so on.

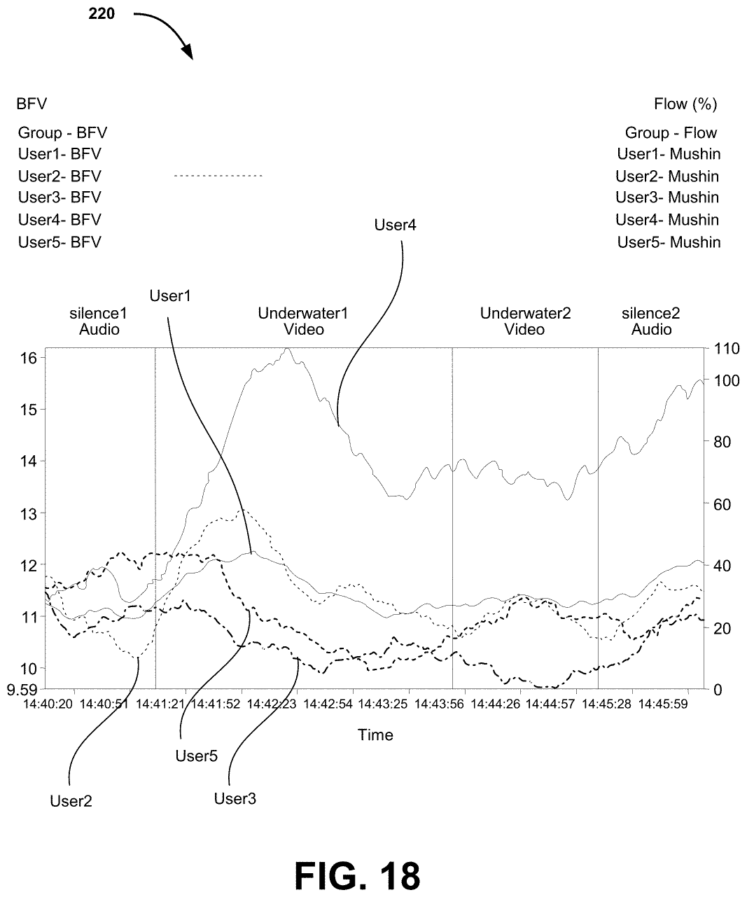

Referring to FIG. 18, an exemplary graphical representation 220 of mean brain frequencies of users (such as User1-User5) are shown compared in order to testify if the users share same state of ANS or same mean brain frequencies during a specific time period (for example, for 5 mins and 39 secs as shown in the graph). As seen, the users (User1-User5) are assumed to together perform a neurofeedback training session using the head gears and initiates the exercise at 14:40:20 and at the end of the session (at 14:45:59), the group flow is compared and it is found that, users (User1, User2, User3 and User 5) share the similar mean frequencies in the range of 10-13 and at almost during entire exercise session, the users (User1, User2, User3 and User 5) are all in nearly the same frequency, however User4 have the mean brain frequency much higher up to 16 and fluctuating a lot during the duration of the session leading to the conclusion that, the user's (User4) state of ANS is not balanced and thus may require some guided interventions or other training sessions to correct the user's (User4) thoughts, perceptions and stimuli that would then effect the brain frequencies and he/she may then be able to match the frequencies of the other users (User1, User2, User3 and User 5).

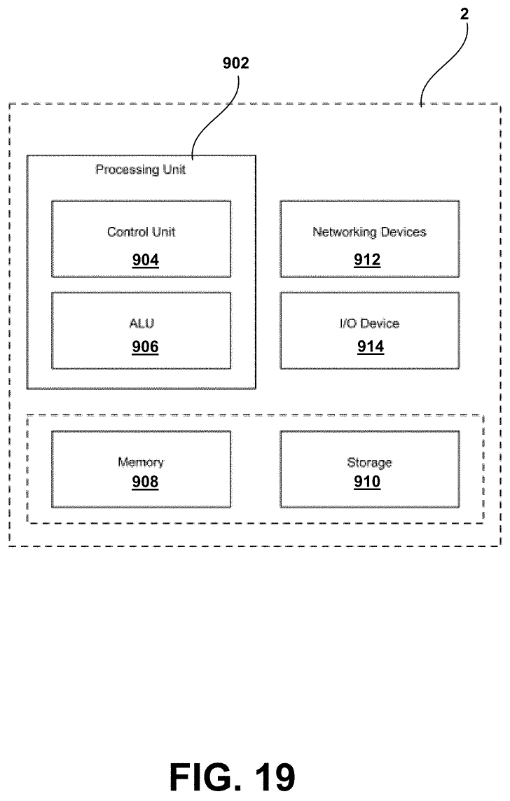

Further, the computing device 2 embodying the neurofeedback program product that to perform above described functionalities or operations may be implemented in the form of mobile phone, computer, laptop and so on. The computing device 2 may include various components as depicted in FIG. 19. FIG. 19 shows the computing device 2 comprises at least one processing unit 902 that is equipped with a control unit 904 and an Arithmetic Logic Unit (ALU) 906, a memory 908, a storage unit 910, a plurality of networking devices 912 and a plurality of input output (I/O) devices 914. The processing unit 902 is responsible for processing the instructions of the schemes. The processing unit 902 receives commands from the control unit 904 in order to perform its processing. Further, any logical and arithmetic operations involved in the execution of the instructions are computed with the help of the ALU 906.

The overall computing environment or computing device 2 can be composed of multiple homogeneous or heterogeneous cores, multiple CPUs of different kinds, special media and other accelerators. The processing unit 902 is responsible for processing the instructions of the schemes. Further, the plurality of processing units 902 may be located on a single chip or over multiple chips.

The scheme comprising of instructions and codes required for the implementation are stored in either the memory unit 908 or the storage 910 or both. At the time of execution, the instructions may be fetched from the corresponding memory 908 or storage 910, and executed by the processing unit 902.

Further, storage 910 may include non-volatile storage elements. Examples of such non-volatile storage elements may include magnetic hard discs, optical discs, floppy discs, flash memories, or forms of electrically programmable memories (EPROM) or electrically erasable and programmable (EEPROM) memories. In addition, the storage 910 may, in some examples, be considered a non-transitory storage medium. The term “non-transitory” may indicate that the storage medium is not embodied in a carrier wave or a propagated signal. However, the term “non-transitory” should not be interpreted that the storage 910 is non-movable. In some examples, the storage 910 can be configured to store larger amounts of information than the memory. In certain examples, a non-transitory storage medium may store data that can, over time, change (e.g., in Random Access Memory (RAM) or cache).

In case of any hardware implementations various networking devices 912 or external I/O devices 914 may be connected to the computing environment to support the implementation through the networking unit and the I/O device unit.

The embodiments disclosed herein can be implemented through at least one software program or program product running on at least one hardware device and performing network management functions to control the elements. The elements shown in the FIGS. 2 through 18 include blocks which can be at least one of a hardware device, or a combination of hardware device and software units.

The foregoing description of the specific embodiments fully reveals the general nature of the embodiments herein that others can, by applying current knowledge, readily modify or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

We claim:

1. A neurofeedback system, comprising:
a head gear comprising
one or more sensors for capturing, in real time, analog neuro-signals from a user's brain, when the user is either at motion or at rest;
one or more Analog to Digital Convertors (ADCs) for converting the captured analog neuro-signals to digital neuro-signals;
at least one transceiver for transmitting and receiving signals to and from the at least one sensor, and the one or more ADCs; and
one or more communication modules in communication with the at least one transceiver for receiving the digital neuro-signals, and in response to receiving the digital neuro-signals transmitting the digital neuro-signals to a neurofeedback program product configured on a computing device communicatively paired with and in communication with the head gear.
Wherein, the neurofeedback program product when run on the computing device performing at least one of:
eliminating, any interference noise present in the received digital neuro-signals arriving at the computing device using one or more specialized filters, to output a set of frequencies,
processing, the outputted set of frequencies to match with a first predefined metric of frequencies classified into a set of standard frequency bands,
averaging, the outputted set of frequencies to derive mean brain frequencies, wherein the mean brain frequencies correspond to homeostasis conditions of the user's Autonomic Nervous System (ANS),
matching, the mean brain frequencies with a second predefined metric of frequencies classified as Brain Frequency Variability (BFV) Indexes, wherein the BFV index correspond to homeostasis conditions of the user's ANS,
displaying, at least one of the mean brain frequencies, and the matching BFV indexes over a Brain Frequency Variability (BFV) Gauge, and
in response to the obtained mean brain frequencies and the matching BFV indexes, providing one or more guided interventions to the user to correct thoughts, perception

2. The neurofeedback system of 1, wherein the analog neuro-signals that are captured from the user's brain correspond to brain's spontaneous electrical activity over a period of time.

3. The neurofeedback system of 1, wherein the set of frequencies ranges from 1-44 Hz.

4. The neurofeedback system of 1, wherein the one or more specialized filters comprises a Dual Parallel Fast Fourier transformation (Dual Parallel FFT) filter.

5. The neurofeedback system of 1, wherein the set of standard frequency bands comprising Delta, Theta, Alpha, Beta, and Gamma.

6. The neurofeedback system of 1, wherein the Brain Frequency Variability (BFV) Indexes ranges from 0-10-0.

7. The neurofeedback system of 1, wherein the one or more guided interventions provided to the user to correct thoughts, perceptions, and stimuli that influences state of the analog neuro-signals of the user's brain comprises brain training exercises, breathing exercises, lifestyle pillars, and physical exercises.

8. The neurofeedback system of 1, wherein the computing device is communicatively paired with and in communication with the head gear using a wireless or a wired network.
